(19) United States
(12) Patent Application Publication
(10) Pub. No.: US 2023/0285136 A1
(43) Pub. Date: Sep. 14, 2023

(54) IMPLANT, MONITORING SYSTEM FOR ORGANISM, AND MANAGEMENT SYSTEM FOR ORGANISM

(71) Applicant: ATONARP INC., Minato-ku, Tokyo (JP)

(72) Inventor: Prakash Sreedhar MURTHY, Minato-ku, Tokyo (JP)

(73) Assignee: ATONARP INC., Minato-ku, Tokyo (JP)

(21) Appl. No.: 18/041,602

(22) PCT Filed: Aug. 20, 2021
(86) PCT No.: PCT/JP2021/030543

§ 371 Date: Feb. 14, 2023

(30) Foreign Application Priority Data
Aug. 20, 2020 (JP) 2020-139398

(51) Int. Cl. A61F 2/06; A61B 5/1455

(52) U.S. Cl. CPC A61F 2/06; A61B 5/1455; A61F 2230/0054

(57) ABSTRACT

There is provided a monitoring system for an organism including an analyzer apparatus and an implant, which is embedded under the skin of the organism, is connected to blood vessels under the skin, and forms a blood flow to an inspection port disposed above the epidermis, within the epidermis, or directly below the epidermis. The analyzer apparatus includes an irradiation apparatus that emits one or more lasers onto the inspection port of the implant embedded under the skin of the organism, and a detector that detects scattered light from the inspection port.

### TECHNICAL FIELD

The present invention relates to an implant to be embedded in an organism, such as a human being, and a system for monitoring and managing the organism using the implant.

### BACKGROUND ART

International Publication WO2014/178199 describes the provision of a monitor for monitoring the internal condition of an organism from the surface of the organism. The monitor includes: a probe including an observation window to be attached to the surface of an organism; a unit for irradiating at least part of an observation area of the organism surface accessed through an observation window with a laser; a unit that detects scattered light 28 caused by the laser irradiation from each of a plurality of observation spots that are spread out in two dimensions in the observation area; a Doppler analyzer unit and SORS analyzer unit for limiting, based on the scattered light obtained from the plurality of observation spots, a first observation spot where it has been determined that scattered light including information on a target portion inside the organism is obtained, out of the plurality of observation spots; and a CARS analyzer unit that acquires a spectroscopic spectrum of at least one component from the first observation spot or observation spots in the periphery, and outputs first information indicating an internal condition of the organism based on the intensity of the spectroscopic spectrum.

### SUMMARY OF INVENTION

When attempting to accurately and non-invasively obtain information inside an organism, especially various information included in blood flowing through a blood vessel, using light such as laser light for example, the effects of absorption and/or scattering of light by the skin and tissue under the skin become problematic. It is therefore desirable to eliminate these effects.

One aspect of the invention is an implant that is embedded under the skin of an organism and includes: connection ends to be connected to a blood vessel under the skin; and an inspection port which is provided on the epidermis, within the epidermis, or just below the epidermis and in which a blood flow is formed via the connection ends. Instead of trying to remove information from the skin and sub-skin tissue, which is included when scattered light from the blood vessels (capillaries) under the skin is acquired, a blood flow is formed above, within, or just below the epidermis using this implant, which makes it possible to eliminate or minimize the effects of absorption and/or scattering due to the skin. This means that it is possible to acquire various information included in the blood flowing through a blood vessel (capillary) with high accuracy by a non-invasive analyzer apparatus that uses one or more lasers.

Although various methods such as infrared absorption spectroscopy can be used as the method of non-invasively detecting components in the blood, Raman spectroscopy is one of the most suitable methods. In order to obtain Raman scattered light, the inspection port may be formed of a member with a high transmissivity for light with wavelengths from red to near-infrared. The implant may be a transparent member such as glass, or may be formed of a flexible member made of resin, such as silicon-based resin or a biomaterial.

Another aspect of the invention is an analyzer apparatus that is attached to skin of an organism, including: an irradiation apparatus (irradiation unit) that irradiates the inspection port of the implant described above that has been embedded under the skin of the organism with one or more lasers; and a detector (detector unit) that detects scattered light from the inspection port. The analyzer apparatus may further include a search apparatus that searches for an inspection port provided on the epidermis, within the epidermis, or just below the epidermis and controls the irradiation location of the lasers. The implant is embedded in the skin so that the inspection port where a blood flow is formed is positioned near the epidermis. Accordingly, there is high probability that it will be possible to visually confirm the inspection port from the skin surface, and the search apparatus may have a function or device of recognizing the inspection port from images. Also, since the inspection port is positioned close to the epidermis, by obtaining scattered light by scanning the vicinity of where the inspection port appears with one or more lasers, it is possible to clearly distinguish scattered light that includes a component corresponding to the blood flow from scattered light that does not contain a component corresponding to the blood flow. Accordingly, the search apparatus may include a function or device that executes preprocessing which acquires scattered light by scanning the vicinity of a position where the inspection port is expected to be disposed with one or more lasers and determines the position of the inspection port from that information.

The irradiation apparatus may include a focusing apparatus (focusing unit) that focuses at least two laser beams, as one example, Stokes light and pump light for generating Raman scattered light, to form a common spot in the inspection port. The analyzer apparatus may also include an optical tweezers apparatus that forms an optical trap inside the inspection port. The analyzer apparatus may further include an electromagnetic field generator that performs microfluidic control on the fluid inside the inspection port.

Another aspect of the invention is a monitoring system for an organism (biological monitoring system, biomonitoring system, biological monitoring apparatus) or a kit for the monitoring system (biological monitoring apparatus kit, biomonitoring kit, apparatus kit or assembly kit) including the implant and analyzer apparatus described above. By embedding the implant in an organism and attaching the analyzer apparatus to the surface (on the skin) of the organism so as to irradiate the inspection port with at least one laser, it is possible to detect various components of the blood accurately and non-invasively. This means that it is possible to provide a monitoring system capable of accurately and continuously monitoring information on the organism without imposing a burden on the organism.

Another aspect of the invention is a management system for an organism (biological management system, biomanagement system biological management apparatus) or a kit for the management system that includes the system for monitoring (or kit for the monitoring system) described above and a dosing apparatus (injection apparatus) that injects a drug into the organism through the skin based on the condition of the organism obtained by the analyzer apparatus. It is possible to provide a management system where by embedding the implant into the organism, attaching the analyzer apparatus to the surface (skin surface) of the organism so as to irradiate the inspection port with at least one laser, and also attaching the dosing apparatus to the skin, it is possible to accurately and continuously monitor information on the organism via various components in the blood non-invasively without imposing a burden on the organism, and based on this, to inject a desired drug in a required amount at the required time into the organism.

Another aspect of the invention is a method of monitoring the condition of an organism. This method includes: embedding an implant under the skin of the organism and connecting to a blood vessel under the skin to form a blood flow in an inspection port provided on the epidermis, within the epidermis, or just below the epidermis; and irradiating the inspection port with at least one laser and detecting scattered light obtained from the inspection port by an analyzer apparatus that has been attached to the surface of the organism (on the skin of the organism). This method may include forming an optical trap inside the inspection port by an optical tweezers apparatus. In addition, the method may include performing microfluidic control of a fluid inside the inspection port by an electromagnetic field generator. The method may also include injecting a drug into the organism via the skin by a dosing apparatus, based on the condition of the organism obtained by the analyzer apparatus.

### BRIEF DESCRIPTION OF DRAWINGS

FIG. 1 is a diagram depicting one example of a management system for an organism attached to the skin.

FIG. 2 is a diagram depicting another example of a management system for an organism attached to the skin.

FIG. 3 is a flowchart depicting the overall operation of a management system for an organism.

### DESCRIPTION OF EMBODIMENTS

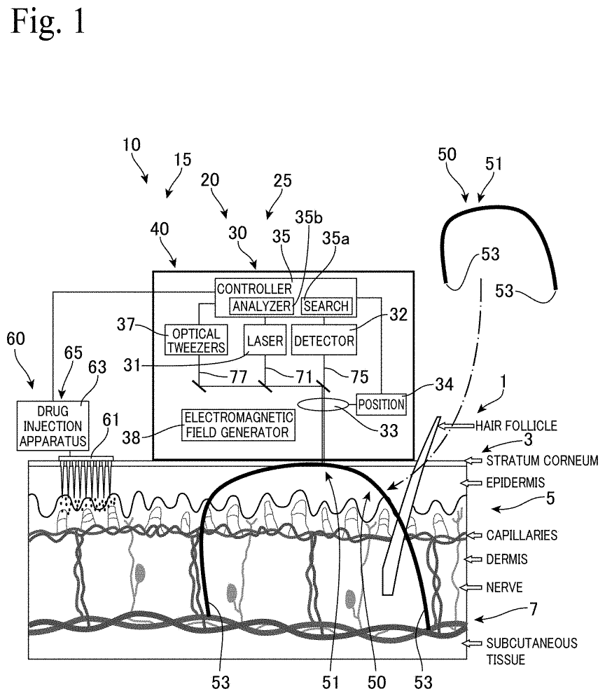

FIG. 1 depicts one example of a system for managing an organism (management system of an organism, biological management system, biomanagement system, health management system) 10 that manages the health condition of an organism (living body), for example, a human body 1. The management system 10 includes a system for monitoring an organism (monitoring system of an organism, biological monitoring system, biomonitoring system) 20 for monitoring the condition of the organism 1 and a dosing system (dosing apparatus) 60 for injecting a drug to maintain the health of the organism 1. The monitoring system 20 is provided by way of a monitoring kit (monitoring kit for the organism, biological monitoring kit, biomonitoring kit) 25 including an implant 50 to be embedded in the organism 1 and an analyzer apparatus (analyzer) 30 that monitors the condition of the organism 1 via the implant 50. One example of the analyzer apparatus 30 is a wearable mobile terminal 40, such as a smart watch, that incorporates a communication function and a user interface. The dosing system 60 is provided by an injection kit 65 including an injector 61 that injects a drug through the skin 5 of the organism 1 and a supplying apparatus (supplier, supplying unit) 63 that supplies a predetermined drug to the injector 61. The management system 10 is provided by a management kit 15 including the monitoring kit 25 and the dosing kit (injection kit) 65.

One example of the implant 50 is an implant (bioport, embedded or embedding body, implant body, implant flow path, or implant port) that is flexible or takes a predetermined shape and is embedded in the skin 5 (that is, under the skin) of the organism 1. The implant 50 includes connecting ends 53 that are connected to one or more blood vessels (blood capillaries) 7 under the skin and an inspection port (inspection window or inspection access portion) 51 provided or appearing (located) above the epidermis 3, within the epidermis 3 or just below the epidermis 3, so that a blood flow is formed in an inspection port 51 via the connecting ends 53. One example of the implant 50 is an artificial blood vessel that is equipped with connecting parts 53 that connect to one or more blood vessels 7 under the skin (as one example, veins and/or arteries in the dermis or subcutaneous tissue) at the ends, has the inspection port 51 in the center, and can be molded or can deform into an arch shape, a U shape, a V shape, or the like. The shape of the inspection port 51 may be a cylindrical shape or may be a flattened shape, may have a shape or a structure that is compatible with irradiation by one or more lasers, and may form an extended shape or a flow path with respect to the end portions 53. The implant 50 may be formed of silicone resin or another resin with high transmissivity for infrared and near infrared, or a suitable biomaterial, such as a material that dissolves or is absorbed into the body.

The implant 50 may be embedded in the organism 1 by a simple operation, or may be inserted from the surface of the organism like a needle used for infusion or a catheter. By forming a blood flow with the implant 50 close to (near) the outside, such as above or below the epidermis 3, the influence of absorption and/or scattering of light for spectroscopic (spectrometric) analysis or absorption analysis due to the skin (such as the dermis or subcutaneous tissue) or the like is eliminated or can be minimized. The implant 50 may be connected to the blood vessel or vessels 7 under the skin, and there are no particular limitations on the embedding location on the organism 1. When the analyzer apparatus 30 is incorporated in a mobile terminal 40, such as a smart watch, the implant 50 may be embedded at a location where the mobile terminal 40 is attached, as one example, on the skin of the wrist where a smart watch is worn.

The analyzer apparatus 30 is a non-invasive analyzer apparatus that uses at least one laser, and by irradiating the inspection port 51 of the implant 50 with at least one laser, a variety of information included in the blood flow artificially formed at the epidermis 3 can be accurately acquired. The analyzer apparatus 30 may use various analytical methods, such as infrared absorption. The analyzer apparatus 30 according to the present embodiment has an irradiation apparatus (irradiation unit) 31, which irradiates the inspection port 51 of the implant 50 with lasers 71, and a detector (detection unit) 32 that detects scattered light 75 from the inspection port 51. The irradiation apparatus 31 includes a focusing apparatus (focusing unit) 33, as one example, an objective lens, which focuses at least two laser beams 71, in the present embodiment, Stokes light and pump light for generating Raman scattered light (CARS light) into a common spot in the inspection port 51. The irradiation apparatus 31 may be an apparatus that emits, as the laser lights 71, probe light in addition to the Stokes light and the pump light.

The focusing apparatus 33 may include an apparatus (irradiation position control apparatus or irradiation position control optical system) 34 that performs control to guide the laser lights 71 to the position of the implant 50 in order to detect (measure) the composition of blood. In order to select an optimal position of the implant 50 to be irradiated with the laser lights 71, the irradiation position control apparatus 34 may further include a function that scans the inspection port 51 of the implant 50 and/or focuses lights on multiple points to select an optimal point. The irradiation position control apparatus 34 may be an apparatus that controls the position and/or orientation of the objective lens 33, or may be an apparatus that controls the irradiation location and/or angle of the lasers by using one or more reflecting apparatuses, such as digital mirror devices.

The implant 50 is embedded in the skin 5 so that the inspection port 51 where a blood flow is formed is located near the epidermis. Accordingly, there is high probability that it will be possible to visually confirm the inspection port 51 from the skin surface, and possible to attach the analyzer apparatus 30 to the skin in adjusting or aligning with the position of the inspection port 51. The analyzer apparatus 30 may further include a search apparatus (or search function) 35a that searches for a specific (detailed) position of the inspection port 51 provided on the epidermis, within the epidermis, or directly below the epidermis and controls the irradiation location of the laser lights 71. One example of the search apparatus 35a may be an apparatus (or function) that acquires an image of the skin surface and recognizes the position of the inspection port, and may be an apparatus that measures blood flow by using the Doppler effect or the like. It is possible to use an image processing function (device) such as OCT, or an apparatus that determines the position of the inspection port 51 by scanning the periphery of the inspection port 51 with the laser beams 71 and judging whether the scattered light 75 contains blood component such as glucose.

The analyzer apparatus 30 includes a search apparatus 35a that accurately detects the position of the inspection port 51 using the laser lights 71. The inspection port 51 is located near the epidermis. This means that the search apparatus (function) 35a can scan the vicinity of a position where the inspection port 51 is expected to appear with the laser lights 71 to obtain the scattered light 75 and clearly distinguish scattered light that includes one or more components corresponding to the blood flow from scattered light that does not contain a component corresponding to the blood flow. The search apparatus 35a may execute processing (preprocessing) that determines the position of the inspection port 51 and decides the irradiation position of the laser beams 71 before the start of measurements, intermittently during measurements, or in parallel with measurements.

An example of a spectrometric (spectroscopic) analysis-type analyzer module (analyzer apparatus) 30 is a Raman analyzer apparatus, and in particular, a CARS (Coherent Anti-Stokes Raman Scattering) analyzer apparatus suited to microscale analysis, an SRS (Stimulated Raman Scattering) analyzer apparatus, or a time-resolved CARS analyzer apparatus, or the like may be used.

The analyzer apparatus 30 includes a controller (control apparatus or control unit) 35 equipped with a function 35b for controlling the irradiation apparatus 31 and analyzing the measurement results for the scattered light 75 obtained by the detector 32, a function 35a for controlling the irradiation apparatus 31 and the detector 32 as a search apparatus, and the like. The controller 35 may further include a function (communication function) that provides the measurement result to the outside, for example an external system, such as a health management server on the cloud, a function for coordinated operation with the dosing system 60, and the like.

The analyzer apparatus 30 may further include an optical tweezers apparatus (optical tweezers) 37 that outputs one or more laser lights 77 that form an optical trap inside the inspection port 51. Optical tweezers are a device that focuses or condenses a laser beam to the utmost limit using an objective lens with a high numerical aperture, and transmit the momentum due to the scattering of photons to generate a force that traps particles of a micrometer-level size. Accordingly, the optical tweezers apparatus 37 can trap particles or molecules of a predetermined size from the blood flowing through the inspection port 51 and target the trapped particles or molecules for Raman spectroscopic analysis.

The analyzer apparatus 30 may further include an electromagnetic field generator apparatus (electromagnetic field generator) 38 that performs microfluidic control over the fluid in the inspection port 51. The electromagnetic field generator 38 includes a function of controlling the flow rate of blood flowing through the implant 50, and may be equipped with a function that keeps the blood flow inside the implant 50 within a range that does not impair the function of the blood vessel or vessels 7 to which the implant 50 is connected and/or may change the blood flow depending on whether measurement is being performed or it is outside a measurement period. Also, through microfluidic control by the electromagnetic field generator 38 and/or the optical tweezers (optical trap), or by performing cooperative control over the apparatuses with the control apparatus 35, it is possible to dynamically form a structure, such as a nano-sized molecular sieve or a nanopen chamber, that captures and/or filters molecules or particles in blood, or the like in the inspection port 51. Accordingly, it is possible to detect the components in the blood steadily, continuously, accurately, and non-invasively without obstructing the blood flow in the blood vessel 7 of the organism 1 and without creating a condition that is harmful to the organism 1, such as a thrombus, within the implant.

Examples of molecules that can be captured by the analyzer 30 via the implant 50 are not limited to blood cells such as red blood cells, white blood cells, lymphocytes, and platelets, and include all molecules that may be present in the blood, such as antibodies, antibody fragments, recombinant antibodies, single-stranded antibodies, receptor proteins, binding proteins, enzymes, inhibitor proteins, lectins, cell adhesion proteins, oligonucleotides, polynucleotides, nucleic acids, and aptamers. The targets of detection and/or identification by the monitoring system 20 including the implant 50 and the analyzer apparatus 30 may be any atom, chemical substance, molecule, compound, composition, microorganism and/or aggregate, and as examples include but are not limited to blood cells, amino acids, peptides, polypeptides, proteins, glycoproteins, lipoproteins, nucleosides, nucleotides, oligonucleotides, nucleic acids, sugars, carbohydrates, oligosaccharides, polysaccharides, fatty acids, lipids, hormones, metabolites, cytokines, chemokines, receptors, neurotransmitters, antigens, allergens, antibodies, substrates, metabolites, cofactors, inhibitors, drugs, pharmaceuticals, nutrients, prions, toxins, poisons, explosive substances, pesticides, chemical warfare agents, biological hazards, radioisotopes, vitamins, heterocyclic aromatic compounds, carcinogens, mutagens, narcotics, amphetamines, barbiturates, hallucinogenic substances, waste, and/or contaminants. Microorganisms may include viruses, bacteria, cells, and the like, but are not limited to such.

This means that by using the biological monitoring apparatus kit (assembly set) 25 including the implant 50 and the analyzer apparatus 30 to embed the implant 50 in the organism 1 and attach the analyzer apparatus 30 to the surface of the organism 1 so that the inspection port 51 can be irradiated with lasers, it is possible to provide the biological monitoring system (monitoring system for the organism) 20 that non-invasively detects various components in blood with high accuracy. In addition, by using the biological monitoring apparatus kit 25, it is possible to provide the biological monitoring system 20 that can accurately and continuously monitor information on the organism 1 without imposing a burden on the organism 1.

The dosing system 60 includes a drug injection apparatus (supply apparatus) 63, which supplies (injects) an amount of one or more drugs required to maintain the health of the organism 1 in one or more predetermined conditions at the required timings based on the measurement results of the analyzer apparatus 30, and an injector 61 that is attached to the skin 5 and injects one or more drug. The injector 61 may use one or more microneedles, or may be a needleless type where drug solutions are introduced through the skin by injection without using a needle.

It is possible to provide a biological management apparatus kit (that is, a set for assembling a biological management apparatus) 15 using the biological monitoring kit 25 and a dosing kit 65 for injecting a drug into the organism 1 through the skin 5 based on the condition of the organism 1 obtained by the analyzer apparatus 30. With this biological management apparatus kit 15, the biological management system (managing system for the organism) 10 can be attached to the organism 1 by embedding the implant 50 into the organism 1, attaching the analyzer apparatus 30 to the surface of the organism 1 (that is, to the skin surface or above the skin) so that the inspection port 51 can be irradiated with lasers, and also attaching the injector 61 of the injection apparatus 60 to the skin 5, and in particular to the epidermis 3. With the biological management system 10, it is possible to accurately and continuously monitor information on the organism 1 via various components in the blood in a non-invasive manner without imposing a burden on the organism 1 and, based on this information, to inject a desired drug into the organism at the required time and in the required amount.

In the biological management system (health management system) 10, the analysis system (monitor) 20 can continuously and accurately measure blood glucose in real time. Accordingly, by using the dosing system 60, it is possible to perform fine control of insulin doses relative to the glucose concentration that is continuously measured. In addition, the biological management system 10 may have a function such as an event recognition module that can predict or grasp the behavior or lifestyle of the organism (human body). The biological management system 10 may detect various actions including the occurrence of events (patient activities) such as exercise and meals, predict the patient's activities using a daily schedule and/or outputs of various sensors, and determine the types and amounts of drugs to be administered, for example insulin, so as to correspond to the patient's predicted condition. This means for example that it is possible to control the blood glucose concentration within a narrow range that has little effect on health. Accordingly, by wearing the biological management system 10, even a diabetic patient can play sports and eat meals in the same way as a healthy person.

The physiologically active substance injected from the injection system (dosing system or drug delivery system) 60 is not limited to insulin, and may be other hormones, prescription drugs, minerals, nutrients, and the like. The biological management system 10 may include a function of determining and controlling the type and amount of medicine (that is, a dosage estimating function). The biological management system 10 may also have a system for sharing real-time biological information and dosing information obtained by the system 10 with an external monitoring system, such as a medical or insurance system, at any time or continuously.

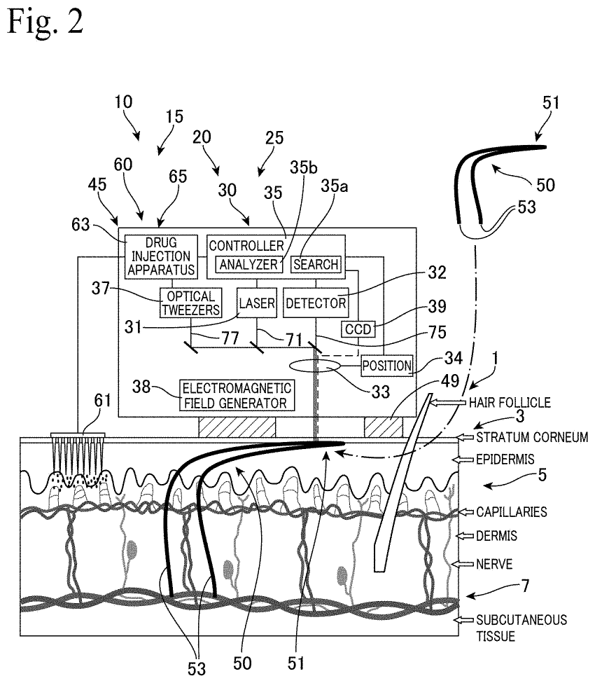

FIG. 2 depicts systems 10 and 20 that use an implant 50 of a different shape. There are no particular limitations on the shape of the implant 50 and by embedding the implant 50 in the skin 5, the implant 50 is connected to a blood vessel or vessels 7 in the skin (or under the skin) or in subcutaneous tissue, and a blood flow, of an amount that reflects information on the organism 1 in real time, is obtained in or near (above or below) the epidermis 3 via the inspection port 51. This means that by using the analyzer apparatus 30, it is possible to analyze components in the blood flow in the inspection port 51 using a non-invasive measurement method (detection method) such as Raman spectroscopy, and thereby accurately measure the state of the organism 1 in real time.

One example of the analyzer apparatus 30 depicted in FIG. 2 is a dedicated terminal 45 for providing the biological management system 10. The dedicated terminal 45 may incorporate functions as the dosing system 60 in addition to functions as the analyzer apparatus 30, and may be attachable to the skin 5 using adhesive pads 49 or the like. There are no limitations on the method of attaching the terminal 45, and any method that enables the terminal 45 to be fixed at a predetermined position on the skin 5 may be used.

This analyzer apparatus 30 includes an imaging element, such as a CCD 39, for image pickup of the appearance of the epidermis 3. The search apparatus 35a may analyze obtained images, determine the detailed position of the inspection port 51, and control the irradiation location of the laser lights 71.

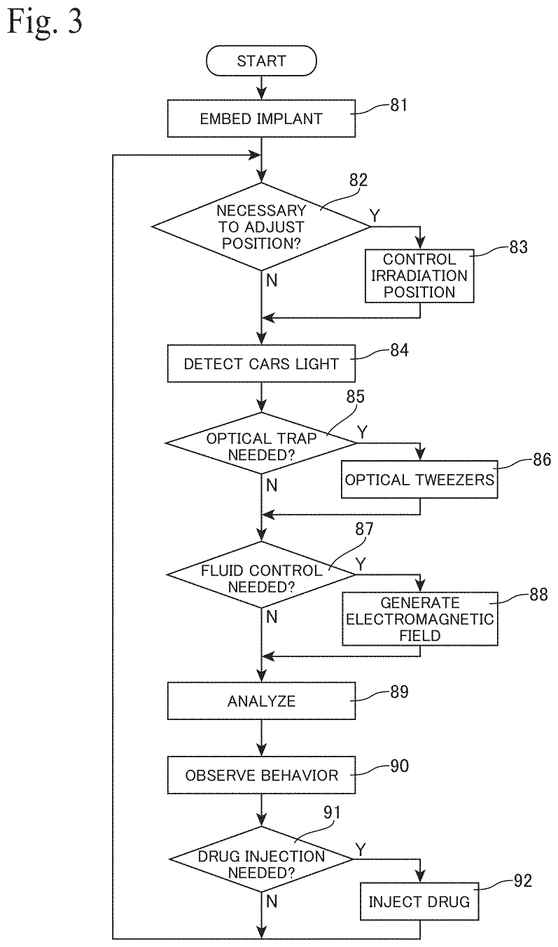

FIG. 3 depicts an overview of a process (method) for monitoring the condition of the organism 1 using the system 10 or 20 described above. First, in step 81, the implant 50 is embedded under the skin (or inside the skin) 5 of the organism 1 to form a blood flow in the inspection port 51 provided above the epidermis, within the epidermis, or directly under the epidermis by connecting both ends 53 of the implant 50 to the blood vessel or vessels 7 under the skin (or inside the skin). Depending on the condition of the patient, and/or the symptoms or condition to be monitored, the implant 50 may be connected between one or more arteries, may be connected between one or more veins, or may be connected between an artery and a vein as the one or more blood vessels 7. The implant 50 may be embedded in advance by a simple operation, or may be installed by the patient using a jig for embedding the implant 50. The implant 50 may be made of a material that dissolves or is absorbed by the organism 1, and a process that embeds the implant 50 may be performed periodically.

In step 84, the inspection port 51 is irradiated with the laser 71 by the analyzer apparatus 30 mounted on an organism surface, for example, the skin 5, and scattered light 75 obtained from the inspection port 51, in the present embodiment, CARS light, is detected. Prior to this process, in step 82, it is possible to search for the position of the inspection port 51 to determine whether adjustment of the irradiation position is necessary, as one example, by analyzing the scattered light 75 in advance. If adjustment of the irradiation position is necessary, in step 83, the detailed position of the inspection port 51 is found using the search apparatus 35a.

In step 84, CARS light 75 is detected and when, in step 85, formation of an optical trap in the inspection port 51 is requested, in step 86, an optical trap is formed by the optical tweezers apparatus 37. Also, in step 87, if it is necessary to control the flow rate inside the inspection port 51, in step 88, an electromagnetic field is formed in the inspection port 51 by the electromagnetic field generator 38 inside the inspection port 51 to perform microfluidic control of the fluid.

In step 89, the CARS light 75 obtained while performing such control on the blood flow formed in the inspection port 51 is analyzed to acquire information relating to the targeted components in the blood. In addition, in step 90, the biological management system 10 may monitor or observe the behavior of the organism (user) 1 based on information from another function of the attached wearable terminal, such as an accelerometer, information from another wearable terminal, information obtained from a server or the like on the Internet (the cloud), or the like.

In step 91, it is determined whether medication is necessary based on the information obtained by the analyzer apparatus 30 and also information obtained by observation of behavior, and in step 92, it is possible to inject medication using the dosing system 60. The dosing system 60 may inject one or more drugs into the organism 1 through the skin using an injection apparatus (injector) 61 based on the condition of the organism 1 obtained by the analyzer apparatus 30.

The method of monitoring the condition of an organism, including the above-described process, may be stored in a computer-readable recording medium and provided as a program (or program product) that controls the monitoring system 20 including the analyzer apparatus 30 and the management system 10. Also, the method of monitoring the condition of an organism may be provided as a program that is downloadable via the Internet or the like, or may be provided as a service via the Internet.

Although specific embodiments of the present invention have been described above, various other embodiments and modifications will be conceivable to those of skill in the art without departing from the scope and spirit of the invention. Such other embodiments and modifications are addressed by the scope of the patent claims given below, and the present invention is defined by the scope of these patent claims.

1. An implant that is configured so that it is able to be embedded under a skin of an organism, the implant comprising an artificial blood vessel that is molded or able to deform into an arch shape, a U shape, or a V shape and the implant includes:
connection ends configured to be connected to a blood vessel of the organism under the skin; and
an inspection port arranged between the connection ends, and which is configured to be provided on an epidermis of the organism, in the epidermis of the organism, or just below the epidermis of the organism, close to an outside of the organism, and in which a blood flow is permitted to flow via the connection ends.

2. The implant according to claim 1,
wherein the inspection port is formed of a member with a high transmissivity of light with a wavelength from red to near infrared.

3. An analyzer apparatus that is configured to be attached to a skin of an organism, comprising:
an irradiation apparatus that irradiates the inspection port of the implant according to claim 1 that has been embedded under the skin of the organism with at least one laser; and
a detector that detects scattered light from the inspection port.

4. The analyzer apparatus according to claim 3,
further comprising a search apparatus that searches for the inspection port provided on the epidermis, within the epidermis, or directly below the epidermis and controls an irradiation location of the at least one laser.

5. The analyzer apparatus according to claim 3,
wherein the irradiation apparatus includes a focusing apparatus that focuses at least two laser beams to form a common spot in the inspection port.

6. The analyzer apparatus according to claim 3,
further comprising an optical tweezers apparatus that forms an optical trap inside the inspection port.

7. The analyzer apparatus according to claim 3,
further comprising an electromagnetic field generator that performs microfluidic control of a fluid inside the inspection port.

8. A monitoring system for an organism comprising:
an implant that is configured so that it is able to be embedded under a skin of an organism, the implant comprising an artificial blood vessel that is molded or able to deform into an arch shape, a U shape, or a V shape and the implant includes:
connection ends configured to be connected to a blood vessel of the organism under the skin; and
an inspection port arranged between the connection ends, and which is configured to be provided on an epidermis of the organism, in the epidermis of the organism, or just below the epidermis of the organism, close to an outside of the organism, and in which a blood flow is permitted to flow via the connection ends; and
the analyzer apparatus according to claim 3.

9. A management system for an organism comprising:
the monitoring system according to claim 8; and
a dosing apparatus that injects a drug into the organism through the skin based on a condition of the organism obtained by the analyzer apparatus.

10. A method of monitoring the condition of an organism comprising:
embedding an implant, which includes an artificial blood vessel with an inspection port and is molded or able to deform into an arch shape, a U shape, or a V shape, under a skin of the organism, and connecting the implant to a blood vessel under the skin to form a blood flow in the inspection port provided on an epidermis of the organism, within the epidermis of the organism, or just below the epidermis of the organism, close to an outside of the organism; and
irradiating the inspection port with at least one laser and detecting scattered light obtained from the inspection port by an analyzer apparatus that has been attached to a skin of the organism.

11. The method according to claim 10,
further comprising searching for the inspection port provided on the epidermis, within the epidermis, or just below the epidermis and controlling an irradiation location of the at least one laser by a search apparatus of the analyzer apparatus.

12. The method according to claim 10,
further comprising forming an optical trap inside the inspection port by an optical tweezers apparatus of the analyzer apparatus.

13. The method according to claim 10,
further comprising performing microfluidic control of a fluid inside the inspection port by an electromagnetic field generator of the analyzer apparatus.

14. The method according to claim 10,
further comprising injecting a drug into the organism through the skin based on a condition of the organism obtained by the analyzer apparatus by a dosing apparatus attached to a surface of the organism.
